(19) United States
(12) Patent Application Publication
(10) Pub. No.: US 2024/0342134 A1
(43) Pub. Date: Oct. 17, 2024

(54) METHODS FOR TREATING DIEASES ASSOCIATED WITH IMPAIRED MEVALONATE PRODUCTION

(71) Applicant: Mor Research Applications Ltd., Ramat Gan (IL)

(72) Inventors: Ohad S. Birk, Rehovot (IL); Yuval Yogev, Givatayim (IL)

(21) Appl. No.: 18/682,201

(22) PCT Filed: Aug. 9, 2022
(86) PCT No.: PCT/IB2022/057432

§ 371 Date: Feb. 8, 2024

Related U.S. Application Data
(60) Provisional application No. 63/231,050, filed on Aug. 9, 2021.

(51) Int. Cl. A61K 31/366; A61K 9/00; A61P 21/00

(52) U.S. Cl. CPC A61K 31/366; A61K 9/0053; A61P 21/00

(57) ABSTRACT

The use of mevalonolactone is provided for treating, preventing or alleviating diseases, disorders or conditions associated, directly or indirectly, with reduced mevalonic acid production in the mevalonate pathway, for example, due to damaged or hindered HMG CoA-reductase enzyme. Oral administration of mevalonolactone is provided for treating muscle cell and/or tissue damage caused at least in part due to impaired mevalonic acid production, as effected in diseases and disorders such as myopathy, statin-associated muscle symptom (SAMS), immune-mediated necrotizing myopathy (IMNM), rhabdomyolysis, muscular toxicity syndrome, myalgia, sarcopenia, and limb girdle muscular dystrophy (LGMD).

### FIELD OF THE INVENTION

The present disclosure relates to treatment of diseases, disorders and conditions associated, directly or indirectly, with impaired mevalonate pathway, particularly, but not exclusively impaired mevalonic acid production in the pathway.

### BACKGROUND

The mevalonate pathway plays a key role in multiple cellular processes by synthesizing sterol isoprenoids such as cholesterol, and non-sterol isoprenoids such as dolichol, heme-A, isopentenyl tRNA and ubiquinone, thereby providing cells with essential bioactive molecules, vital in multiple cellular processes. This pathway converts mevalonate into sterol isoprenoids (e.g., cholesterol), which are indispensable precursors of bile acids, lipoproteins, and steroid hormones, and into a number of hydrophobic molecules, nonsterol isoprenoids. These intermediates of the mevalonate biosynthetic pathway play important roles in the post-translational modification of a multitude of proteins involved in intracellular signaling and are essential in cell growth/differentiation, gene expression, protein glycosylation and cytoskeletal assembly.

Cholesterol is the end product of the mevalonate pathway. In this pathway, three molecules of acetyl-CoA condense successively to form (3S)-hydroxy-3-methylglutaryl-CoA (HMG-COA). This CoA derivative is reduced to mevalonate by HMG-COA reductase, the rate-limiting enzyme of the mevalonate pathway in humans. Its specific inhibitors, pravastatin and related compounds, are widely used as cholesterol-lowering agents. Mevalonate is then phosphorylated twice and decarboxylated to form isopentenyl diphosphate (IPP). IPP is then converted to its isomer, dimethylallyl diphosphate (DMAPP), catalyzed by IPP isomerase. IPP and DMAPP synthesized in the mevalonate pathway are used as basic units in the biosynthesis of isoprenoids such as sterols, carotenoids, and dolichols.

The enzyme 3-hydroxy-3-methyl-glutaryl-coenzyme A reductase (HMGCR) catalyzes the conversion of HMG-COA to mevalonic acid, the rate-limiting step in the synthesis of cholesterol and other isoprenoids in the mevalonate pathway. Thus, it plays a critical role in cholesterol homeostasis. The gene coding for the enzyme is designated HMGCR. Normally, in mammalian cells this enzyme is highly regulated, and its effect are strictly controlled. This enzyme is the target of the widely available cholesterol-lowering drugs known collectively as statins, which help treat dyslipidemia.

Myopathy is the main adverse effect of statins that act through inhibiting HMG CoA-reductase. The mechanism of statin myopathy and its treatment are yet to be resolved.

### SUMMARY

The present disclosure is based on a discovery by the present inventors that a homozygous loss of function, missense mutation in enzyme 3-hydroxy-3-methylglutaryl-coenzyme A (HMG CoA) reductase gene (HMGCR), the gene encoding the enzyme HMG CoA-reductase, caused adult-onset severe progressive limb girdle myopathy, which was effectively treated with mevalonolactone. The present inventors synthesized and purified, through a biochemical process, the biologically active stereoisomer D-mevalonolactone (R-(−)-mevalonolactone) and have demonstrated its safety in mice. Following these achievements, the present inventors attempted oral treatment, for the first time, of patients afflicted with HGMCR myopathy, and have successfully demonstrated clinical efficacy in alleviating the limb girdle muscular dystrophy caused by biallelic HGMCR mutation without significant adverse effects.

Furthermore, it was shown by the present inventors that oral mevalonolactone administration resolved statin-induced myopathy in mice.

In one aspect, the present disclosure relates to methods for treating, preventing or alleviating at least one of:
(i) impaired mevalonic acid production;
(ii) cell and/or tissue damage caused at least in part due to impaired mevalonate pathway;
(iii) cell and/or tissue damage caused at least in part due to impaired mevalonic acid production;
(iv) a disease disorder or condition associated with impaired mevalonate pathway;
(v) a disease disorder or condition associated with impaired mevalonic acid production;
(vi) a disease, disorder or condition associated with impaired function of the enzyme 3-hydroxy-3-methyl-glutaryl-coenzyme A reductase (HMGCR); and/or
(vii) a disease, syndrome, disorder or condition associated with statin-based therapy.

Any of the disclosed methods comprises administering a therapeutically effective amount of mevalonolactone to a subject in need thereof.

In some embodiments, a contemplated treatment comprises oral administration of mevalonolactone, particularly, but not exclusively, the biological active isomer D-mevalonolactone.

In some embodiments, the damaged cell or tissue treated is muscle cell or muscle tissue.

In some embodiments, the impaired mevalonate pathway or the impaired mevalonic acid production is associated with impaired function of the enzyme HMGCR, which accounts for the natural production of mevalonic acid in the body.

The impaired function of HMGCR enzyme may be due to inhibition, suppression or arrest of its activity, for example, by one or more deleterious and/or harmful, direct or indirect, effects of exogenous and/or endogenous agents, circumstances or events (i.e., reactions). Exogenous agents may be one or more toxins, medications or a combination thereof, for example, checkpoint inhibitors, immunotherapy, corticosteroids, cholesterol-lowering drugs, amiodarone, colchicine, chloroquine, antivirals and protease inhibitor, omeprazole, or any combination thereof.

In some embodiments, the medicament is a statin.

The endogenous events may be mutations, post transcriptional modifications, protein post translational modifications and/or a chemical or biological agents, reactions or processes in the body.

In some embodiments, the function of HMGCR enzyme is inhibited, hindered or suppressed due to one of more mutations in the gene that encodes the enzyme (HMGCR).

The disease, syndrome, disorder or condition treatable by any of the disclosed methods is at least one of: myopathy, statin-associated muscle symptom (SAMS), immune-mediated necrotizing myopathy (IMNM), rhabdomyolysis, muscular toxicity syndrome, myalgia, sarcopenia, limb girdle muscular dystrophy (LGMD) or biallelic HGMCR mutation limb girdle muscular dystrophy (HGMCR-LGMD).

In a further aspect, the present disclosure relates to biological, fermentation-based process for producing D-mevalonolactone.

### BRIEF DESCRIPTION OF THE DRAWINGS

Some embodiments are herein described, by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced.

In the drawings:

FIG. 1 is a pedigree scheme of a Bedouin kindred from the Negev area of Israel indicating 6 individuals affected by adult-onset limb girdle muscular dystrophy (LGMD);

FIG. 2 is a graph showing the time course of an exemplary batch fermentation process for production of mevalonolactone. Induction with IPTG was performed at Time=0;

FIGS. 3A-3E are spectra obtained by various analysis methods employed in assessment of mevalonolactone purity. (3A): gas chromatography-mass spectrometry (GC-MS) chromatogram of a sample produced using batch fermentation. (3B) Spectra of the 5.036 peak, showing the expected fragments at 43, 71 and 58 m/z. (3C) GC-MS reference spectra for mevalonolactone, obtained from AIST SDBS. (3D-3E) Observed 1H and 13C-NMR spectra of samples (up) and expected reference spectra obtained from AIST SDBS (bottom);

FIGS. 4A-4B are schematic illustration of D-mevalonolactone treatment protocol of a HMGCR mutation-associated limb girdle muscular dystrophy (LGMD) patient. (4A): intended treatment regimen and timeline, lasting one year, with an escalating dose protocol lasting 2 months. (4B): actual treatment regimen and timeline preformed, modified due to incident events, likely not related to the treatment: (a) subjective feeling of swelling in the wrists (no measured difference), (b) severe headache, resolved overnight (c) near syncope;

FIG. 5 is a graph showing the activity of wild type (WT) and mutant HMG-COA reductase (HMGCR) activity as a function of concentration as measured in spectrophotometric NADPH oxidation assay;

FIGS. 6A-6C are curves of isothermal calorimetric (ITC) analysis of the thermodynamics of wild type (WT) and mutated HMG-COA reductase reaction to a known inhibitor pravastatin. (6A): WT versus control. (6B): Mutant versus control. (6C) WT versus mutant;

FIG. 7 is a graph showing the plasma mevalonolactone levels of a patient afflicted with LGMD associated with HMGCR mutation (patients V: 2) (n=20 on multiple occasions), and healthy individuals (n=10), normalized to average level of healthy individuals;

FIGS. 8A-8C are graphs showing the evaluation of muscle strength and lung functions throughout a treatment period of a patient afflicted with LGMD associated with HMGCR mutation (patient V: 2), orally treated with mevalonolactone. (8A): evaluation of muscle strength by dynamometry (top) and manual muscle test (MMT) by an experienced neurologist (bottom). (8B): evaluation of distal muscles by an experienced neurologist. (8C): lung functions assessed by spirometry; and

FIGS. 9A-9C are graphs showing the effect of oral mevalonolactone treatment in statin-induced myopathy in mice. Mice were treated daily with injections of either cerivastatin, simvastatin or 0.9% saline solution daily for 14 days, with or without 200 mg/kg oral mevalonolactone. Mice were housed in PhenoMaster cages for the last 3 days of treatment. (9A): grip strength test on day 14. (9B): hanging wire test on day 14. (9C): hanging wire test throughout the study.

### DETAILED DESCRIPTION

A novel form of autosomal recessive limb girdle muscular dystrophy (LGMD) caused by a partial loss-of-function missense mutation in HMGCR, which encodes HMG CoA-reductase (HMGCR) a key enzyme in the mevalonate pathway, was discovered by the present inventors. This disease is referred to herein as HMGCR-LGMD. While genome-wide association studies (GWAS) showed HMGCR polymorphisms to be associated with total cholesterol and lipoprotein levels and response to statin use, this is the first finding of a human disease caused by mutation in HMGCR. Genetic perturbations in non-human HMGCR orthologs have been associated with severe phenotypes.

Pharmacological inhibition of HMG CoA-reductase with drugs of the statin class is associated with myalgia and myopathy, up to rhabdomyolysis, which is potentially fatal. The HMGCR-LGMD patients presented with a syndrome that shows a high overlap with statin-induced myopathy. Moreover, intermediate to severe statin associated muscle symptoms (SAMS) usually present with proximal muscle weakness, further delineating the similarity to HMGCR-LGMD. Chronic or subacute presentation of SAMS or severe statin myopathy may mimic LGMD.

The present inventors envisioned a critical role for HMG CoA-reductase activity in the etiology of HMGCR-LGMD and SAMS and have successfully demonstrated the feasibility of oral mevalonolactone treatment. It is noted that mevalonolactone has never been used to treat human patients, and so there is no available knowledge as to its safety, dosage and efficacy. As such, the present inventors carried a study limited to a single patient suffering from a very severe form of HMGCR-LGMD, where no treatments are available. The contribution of mevalonolactone treatment to this patient was notable, with dramatic improvement in daily function.

Furthermore, the present inventors successfully demonstrated that in a murine model, treatment with mevalonolactone protected from the myotoxicity induced by high dose statin treatment.

In a broad aspect, the present disclosure relates to treating diseases, disorders or conditions associated with impaired or damaged mevalonate pathway in a subject.

Embodiments of the present disclosure relates to treating diseases, disorders or conditions associated with impaired, disturbed or otherwise damaged or destructed mevalonic acid production in a subject.

Some embodiments of the disclosure pertain to curing, preventing, ameliorating and/or alleviating cell and/or tissue damage, for example, damage to muscle cell or muscle tissue, caused at least in part due to impaired mevalonate pathway and/or impaired mevalonic acid production.

The mevalonate pathway leads to the synthesis of sterols and isoprenoids. The first committed step of the mevalonate pathway is conversion of 3-hydroxy-3-methylglutaryl-CoA (HMG-COA) to mevalonic acid (mevalonate) by HMG-COA reductase (HMGCR) in the presence of nicotinamide adenine dinucleotide phosphate (NADPH). In the next step of the pathway, mevalonate is metabolized to isopentenyl pyrophosphate (IPP) and dimethylallyl pyrophosphate (DMAPP). Farnesyl pyrophosphate synthase catalyzes sequential condensation reactions of DMAPP with two units of IPP to form farnesyl pyrophosphate (FPP) and geranylgeranyl pyrophosphate synthase catalyzes yet another condensation reaction to form geranylgeranyl pyrophosphate (GGPP). FPP is the branch point for several pathways leading to various end-products including cholesterol, steroid and dolichols. Additionally, FPP can also be converted into geranylgeranyl pyrophosphate (GGPP) by GGPP synthase. Squalene synthase catalyzes the first reaction of the pathway committed exclusively to cholesterol biosynthesis and plays a crucial role in directing intermediates to either sterol or non-sterol branches of this metabolic pathway.

HMG-COA reductase, the rate-limiting enzyme for the mevalonate pathway and is one of the most finely regulated enzymes. Regulation begins at the transcriptional level; if cholesterol or other sterol isoprenoids are in shortage, sterol regulatory element binding proteins (SREBP) are activated and they bind to sterol regulatory elements (SREs) present on the HMGR promoter, increasing its transcription. Cholesterol also regulates the degradation of HMGR, promoting its association with gp78, an ubiquitin-E3 ligase that directs the enzyme towards proteasome 26s. HMGR is also regulated at the post-translational level, by phosphorylation mediated through AMP-activated protein kinase (AMPK). This enzyme is sensitive to the AMP: ATP ratio, and is activated by increased AMP concentration, thus in case a of metabolic stress, it deactivates HMGR, reducing cellular metabolism.

The mevalonate pathway generates a key intermediate for cholesterol production, a fundamental constituent of cell membranes. Moreover, cholesterol is also converted to steroid hormones, regulating different cellular pathways, vitamin D and bile acid production. IPP is the first step also in other, non-cholesterol, reactions; it is important for the production of farnesyl-pyrophosphate (FPP). FPP is converted in dolichols, used to assemble carbohydrate chains in glycoproteins, or in ubiquinones (or coenzyme Q10), electron transporters in mitochondria; or it is used to farnesylate or geranylate proteins, thus targeting them to cell membranes. In summary, the mevalonate pathway is responsible for numerous cellular processes and the key enzymes described above undergo different regulation to maintain a constant supply of IPP.

The term “impaired mevalonate pathway”, as used herein, refers to hindered, deficient, damaged, disabled, or otherwise partially or completely non-functioning one or more enzymatic reaction in the chain of reactions in the pathway. Impaired mevalonate pathway also encompasses such circumstances wherein one or more substrates of enzymatic reaction of the pathway are decreased, compromised, deficient, damaged, insufficient, lessen or sparse.

In some embodiments, the impaired mevalonate pathway or impaired reactions, enzymes and/or substrates are upstream of the mevalonic acid productions step. In accordance with these embodiments, impairment as, defined herein, is of the “upper mevalonate pathway”.

The term “impaired mevalonic acid production”, as used herein, refers to decreased, compromised, deficient, damaged, insufficient, lessen, hindered, disabled, sparse, or otherwise inadequate production of mevalonic acid in the body, which causes, prone to cause, leads or may lead, directly or indirectly, to a state of illness, or otherwise negatively affects or threatens the health or wellbeing of a subject. Such impaired mevalonic acid production may result in physiological mevalonic acid levels or amounts which deviate from normal levels or amounts. For example, the impaired mevalonic acid production may account for reduced, diminished, or decreased level of mevalonic acid, or even nulled production compared to a normal physiological state, when normal ranges of mevalonic acid are considered from about 2 ng/ml to about 10 ng/ml. A physiological situation in which the level of mevalonic acid is not in the normal range, as well known in the art, is interchangeably referred to herein as “unfavorable mevalonic acid level”, or “harmful mevalonic acid level”. In the context of embodiments described herein, unfavorable or harmful mevalonic acid level is below normal levels, for example substantially below normal levels, e.g., at least 20%, at least 30%, at least 40% or at least 50% below normal levels.

The term “illness” as used herein refers to a condition in which the body or mind is harmed because an organ or part is unable to work as it usually does. In the context of embodiments of the present disclosure, illness is the reaction of the body to a disease, disorder, syndrome, medication side effects, or any physical condition that are affected, directly or indirectly, by impaired mevalonic acid production.

In some embodiments, the present disclosure relates to a method for treating, alleviating or preventing unfavorable or harmful mevalonic acid levels in a subject, the method comprises administration of a therapeutically effective amount of mevalonolactone to a subject in need thereof.

In some embodiments, the present disclosure relates to a method for treating, ameliorating or preventing a disease disorder or condition associated with impaired mevalonic acid production, wherein the method comprises administration of a therapeutically effective amount of mevalonolactone to a subject in need thereof.

The term “disease disorder or condition associated with impaired mevalonic acid production”, as used herein, refers to any and all diseases, disorders, conditions and/or medication side effects caused directly or indirectly by impaired mevalonic acid production and/or by unfavorable or harmful mevalonic acid level in the body. This term includes a disease, disorder or condition developed in a secondary stage, a complication and/or a synchronous or asynchronous sequela of impaired mevalonic acid production and/or of unfavorable or harmful mevalonic acid level in the body whereby, optionally, the disease, disorder, medication side effects, or condition worsen, exacerbates or progresses due to impaired mevalonic acid production and/or due to unfavorable or harmful mevalonic acid level in the body.

In some embodiments, the disease, disorder of condition treatable by a contemplated method is the pathologic consequences of, or associated with, impaired function of the enzyme 3-hydroxy-3-methyl-glutaryl-coenzyme A reductase (HMGCR), particularly, but not exclusively, wherein the activity is impaired mevalonic acid production.

Thus, in some embodiments, the present disclosure relates to a method for treating, preventing or ameliorating a disease, disorder, medication side effects or any other condition associated with impaired function of the enzyme HMGCR, which results in impaired mevalonic acid production. The method comprises administration of a therapeutically effective amount of mevalonolactone to a subject in need thereof. The impaired function of the enzyme HMGCR may be, for example, conversion of (3S)-hydroxy-3-methylglutaryl-CoA (HMG-COA) to mevalonic acid in the mevalonate pathway.

The impaired function of HMGCR may be due to inhibited, suppressed or arrested activity of the enzyme. For example, the enzyme may be inhibited or suppressed by the action or occurrence of endogenous and/or exogenous agents, conditions and/or events.

Endogenous conditions or event that may affect the function and integrity of the enzyme HMGCR, include, but are not limited to, mutations, for example one or more mutations in the gene that encodes the enzyme, namely the gene HMGCR, post transcriptional modifications, protein post translational modifications and/or any chemical or biological agent or factor (e.g., cytokines, chemokines) produced in the body, which interfere (e.g., inhibit, arrest, eradicate) the synthesis and/or action of one or more enzymes in the upper mevalonate pathway.

The term “protein post-translational modification” refers to a modification that, e.g., increases the functional diversity of the proteome by the covalent addition of functional groups or proteins, proteolytic cleavage of regulatory subunits, or degradation of entire protein. Such modification includes, for example, one or more of phosphorylation, glycosylation, ubiquitination, nitrosylation, methylation, acetylation, lipidation and proteolysis and influence almost all aspects of normal cell biology and pathogenesis.

The term “post-transcriptional modification” or “co-transcriptional modification”, as used herein are interchangeable and refer to a set of biological processes common to most eukaryotic cells by which an RNA primary transcript is chemically altered following transcription from a gene to produce a mature, functional RNA molecule that can then leave the nucleus and perform any of a variety of different functions in the cell. There are many types of post-transcriptional modifications achieved through a diverse class of molecular mechanisms. For example, the conversion of precursor messenger RNA (mRNA) transcripts into mature mRNA that is subsequently capable of being translated into protein. This process includes three major steps that significantly modify the chemical structure of the RNA molecule: the addition of a 5′ cap, the addition of a 3′ polyadenylated tail, and RNA splicing. Such processing is vital for the correct translation of eukaryotic genomes because the initial precursor mRNA produced by transcription often contains both exons (coding sequences) and introns (non-coding sequences); splicing removes the introns and links the exons directly, while the cap and tail facilitate the transport of the mRNA to a ribosome and protect it from molecular degradation.

Post-transcriptional modifications may also occur during the processing of other transcripts which ultimately become transfer RNA (tRNA), ribosomal RNA, or any of the other types of RNA used by the cell.

In some embodiments, the impaired function of HMGCR is due to one or more mutations in the gene coding for the enzyme (HMGCR).

The term “mutation”, in broad sense, describes changes or modifications in DNA or protein sequence compared with a reference sequence. As used herein, mutation is the permanent alteration of the nucleotide sequence of the genome of an organism, virus, or extra chromosomal DNA or other genetic elements. Mutations in the structure of genes can be classified as small-scale mutations or large-scale mutations. Small-scale mutations are types of gene mutations, such as those affecting a small gene in one or a few nucleotides, for example, point mutations. A mutation is said to be punctual when it touches one or more nucleotides of the same gene. Point mutations include substitution mutations, which in turn includes missense mutations, nonsense mutation and silent mutations.

A missense mutation is a point mutation that results in the replacement of one nucleotide by another. In some cases, this change causes a change in the amino acid encoded, which may or may not have an impact on the function of the protein produced by the gene in the case of a gene encoding, or the affinity for a transcription factor, in the case of a promoter region of the DNA.

In nonsense mutation, instead of substituting one amino acid for another, however, the altered DNA sequence prematurely signals the cell to stop building a protein. This type of mutation results in a shortened protein that may function improperly or not at all.

Silent mutations are mutations that do not alter the sequence of a protein because of the redundancy of the genetic code (the new triplet codes for the same amino acid as the original triplet), or because it affects an area not coding DNA or an intron.

Large-scale mutations include deletions, modifications or structural variations of DNA sequences that hinder or modify the generation of the wildtype sequence of the mRNA and/or protein encoded by the gene, resulting in a modified sequence or production levels of the encoded mRNA and/or protein.

Mutations result either from inherited conditions or from accidents during the normal chemical transactions of DNA, often during replication, or from exposure to high-energy electromagnetic radiation (e.g., ultraviolet light or X-rays) or particle radiation or to highly reactive chemicals in the environment.

The terms “mutant” and “variant”, as used herein, are interchangeable and refer to an organism (e.g., a human being) that is different from others of its type because of a permanent change in its genes (i.e., mutation).

Allele, also called allelomorph, is any one of two or more genes that may occur alternatively at a given site (locus) on a chromosome. Allele is a genetic locus or the base sequence on the chromosome. Alleles may occur in pairs, or there may be multiple alleles affecting the expression (phenotype) of a particular trait. The combination of alleles that an organism carries constitutes its genotype. If the paired alleles are the same, the organism's genotype is said to be homozygous for that trait; if they are different, the organism's genotype is heterozygous. A dominant allele will override the traits of a recessive allele in a heterozygous pairing.

All genetic traits are the result of the interactions of alleles. Mutation, crossing over, and environmental conditions selectively change the frequency of phenotypes (and thus their alleles) within a population.

Biallelic mutation is a mutation that occurs on both alleles of a single gene. When used to refer to pathogenic variants, biallelic refers to a pathogenic variant affecting both alleles (homozygous for the same pathogenic variant at both alleles, or compound heterozygous with a different pathogenic variant at each allele). Monoallelic refers to a genotype affecting only one allele (heterozygous).

In some embodiments, a contemplated method is applied in the treatment, prevention or amelioration of a disease, disorder, syndrome or condition associated with impaired function of the enzyme HMGCR, due to one or more genetic modifications (i.e., mutations) in the HMGCR gene. In some embodiments, the disease, disorder or syndrome is limb girdle muscular dystrophy (LGMD).

Limb girdle muscular dystrophy is a broad term encompassing many rare autosomal diseases that share proximal muscle weakness as the major common attribute, with different genetic bases and underlying pathophysiological processes. Disease is usually progressive to a variable degree, ranging from minor disability to complete inability to ambulate, and can involve the large proximal muscles, as well as axial and facial muscles. Different forms of LGMD may exhibit skeletal muscle hypertrophy, kyphoscoliosis, and contractures, or involve other muscle groups and manifest with muscle weakness, cardiomyopathy, dysphagia, and respiratory difficulties. Other features may include mental retardation, seizures, arrythmia and ocular anomalies. The age of onset varies greatly, from early infancy to late-adult onset. Most LGMDs present with elevated creatinine kinase (CK) and myopathic electromyographic features. LGMD syndromes show a wide range of genetic heterogeneity, with over 50 genetic loci and 30 known disease associated genes, further complicating diagnosis. The pathological processes underlying most LGMDs can be broadly linked to disorders in structure of the sarcolemma, integration with extracellular matrix, nuclear or plasma membrane stability, or mechanical sensing and signal transduction. Currently, there is no available treatment for any of the LGMDs other than supportive care, but several trials of genetic therapy are ongoing.

The present inventors discovered a novel form of LGMD caused by HMGCR mutation, which effects the molecular pathway of statin-induced myopathy. As shown in the Examples section herein, the present inventors successfully treated a human patient diagnosed with this novel form of LGMD by oral administration of D-mevalonolactone.

Some embodiments of the present disclosure relate to a method for curing, preventing and/or alleviating a disease, disorder, syndrome, medication side effects and/or any other condition associated with damage to muscle cells or muscle tissue, caused at least in part by impaired mevalonic acid production, wherein the method comprises administration a therapeutically effective amount of mevalonolactone.

In some embodiments, the disease or disorder is myopathy.

The term “myopathy”, as used herein, refers to diseases that affect skeletal muscles, e.g., attack muscle fibers and weaken muscles. Myopathies are neuromuscular disorders in which the primary symptom is muscle weakness due to dysfunction of muscle fiber. Other symptoms of myopathy include muscle cramps, stiffness, and spasm. Myopathies can be inherited (such as the muscular dystrophies) or acquired.

Inherited myopathies arise by inheriting a gene mutation from a parent that causes the disease. Inherited myopathies include, but are not limited to, congenital myopathies: characterized by developmental delays in motor skills. Skeletal and facial abnormalities are occasionally evident at birth; muscular dystrophies: characterized by progressive weakness in voluntary muscles and progressive degeneration of muscle tissue due to abnormal or insufficient structural support proteins being present. Sometimes evident at birth; mitochondrial myopathies: caused by genetic abnormalities in mitochondria, cellular structures that control energy, include Kearns-Sayre syndrome, MELAS and MERRF.

Acquired myopathies develop later in life and can be due to other medical disorders, infections, exposure to certain medications or electrolyte imbalances, among other possibilities. Toxic myopathy happens when a toxin (e.g., alcohol, toluene) or medication interferes with muscle structure or function. Such medications include, for example, checkpoint inhibitor immunotherapy (pembrolizumab, nivolumab), corticosteroids (prednisone), cholesterol-lowering drugs (e.g., statins), amiodarone, colchicine, chloroquine, antivirals and protease inhibitors used in the treatment of HIV infection, omeprazole, or any combination thereof. For example, myopathy is the main adverse effect of HMG CoA reductase inhibitors, commonly known as statins, one of the most prescribed medication classes in the western world.

The prognosis for individuals with a myopathy varies. Some individuals have a normal life span and little or no disability. For others, however, the disorder may be progressive, severely disabling, life-threatening, or fatal.

Treatments for the myopathies depend on the disease or condition and specific causes. Supportive and symptomatic treatment may be the only treatment available or necessary for some disorders. Treatment for other disorders may include drug therapy, such as immunosuppressives, physical therapy, bracing to support weakened muscles, surgery or termination of treatment with a drug whose adverse effect is myopathy.

As shown in the Examples section herein, the present inventors successfully treated statin-induced myopathy in mice with this active agent, with no evident adverse effects.

In some embodiments, the present disclosure relates to a method for treating, preventing or ameliorating a disease, disorder, syndrome or condition associated with impaired mevalonate pathway or impaired mevalonic acid production caused by exogenous agents, events and/or circumstances. Such exogenous agents, events and/or circumstances may be external agents and/or environmental conditions, either alone or in any combination thereof, that have deleterious and/or harmful, direct or indirect, effects, e.g., on production and/or function of any one or more of the enzymes and/or substrates in the mevalonate pathway, for example, the upper mevalonate pathway.

For example, certain external agents and/or environmental conditions such as diet, activity level and/or drug exposure may negatively affect the function of HMGCR in reducing (3S)-hydroxy-3-methylglutaryl-CoA (HMG-COA) to mevalonic acid and/or may damage the substrate itself (mevalonic acid).

The principal compounds that induce exogenous mevalonate pathway blockade are the statins, which are a class of compounds that act as competitive inhibitors of 3HMG-COA reductase. Statins, in general, are able to bind to a portion of HMG-COA binding site, thus blocking the access of this substrate to the active site of the enzyme; effectively reducing the rate of mevalonate productions.

Statins are classified into statins produced by fungi (such as Lovastatin, Simvastatin) and statins synthetically made (such as Atorvastatin, Fluvastatin). All statins share a conserved HMG-like moiety covalently linked to a more or less extended hydrophobic group.

By blocking HMG-COA reductase, statins induce a decrease in cholesterol level and simultaneously other by-products of the mevalonate pathway such as farnesyl pyrophosphate (FPP), geranylgeranyl pyrophosphate (GGPP), dolichols and coenzyme Q10. Inhibition of HMG-CoA reductase has a pleiotropic effect, due to the different affinities of key enzymes in the mevalonate pathway. FPP, the main metabolite in this pathway, could be converted to cholesterol through squalene synthase and this enzyme has a Kₘ for the substrate of about 2 μM. GGPP synthase, instead, could convert FPP to GGPP, with a Kₘ of 1 μM; GGPP is attached to different proteins (the majority of which pertain to the Rab family) to ensure their correct localization. On the other hand, protein farnesyl trasferase (FTase) uses FPP to attach a farnesyl group to specific proteins, such as the family of small GTPase proteins (Ras and Rho GTPases), with a Kₘ of 5 nM. Therefore, inhibition of HMG-COA reductase lowers FPP levels and the first consequence is a reduction in cholesterol levels; following that, GGPP levels are reduced, causing mislocalization and loss of activity of specific proteins.

Statins are a lipid-lowering drug class, serving as first-line treatment for dyslipidemia, effective in decreasing the incidence of adverse cardiovascular events including myocardial infarct and stroke. Up to 30% of statin users suffer from statin associated muscle symptoms (SAMS), rarely causing life threatening rhabdomyolysis or immune mediated necrotizing myositis (IMNM).

Mevalonate pathway blockade caused by treatment with statins has been linked to mitochondrial dysfunction, specifically by lowering mitochondrial membrane potential and increasing release of pro-apoptotic factors. Strong oxidative stress, which induces mitochondrial dysfunction, could be due to the action of statins on the mevalonate pathway, which decrease levels coenzyme Q10 and dolichol considered as anti-oxidants defense systems.

Coenzyme Q10 (CoQ10) is a product of the mevalonate pathway and is an important electron transporter of the mitochondrial respiratory chain. A decrease in coenzyme Q10 levels, caused by mevalonate pathway blockade, could result in an abnormal mitochondrial respiratory function causing mitochondrial and oxidative damage.

Dolichol, a polyprenol compound, is an important free-radical scavenger in cell membranes. A significant decrease in dolichol levels is observed after lovastatin administration in in vivo models; a lack of this compound might cause oxidative stress and mitochondrial damage.

Nevertheless, mitochondrial dysfunction caused by statins could also be related to a decrease in prenylated protein levels; indeed, statins treatment could lead to a reduction in cholesterol level, and in farnesyl pyrophosphate (FPP) and in geranylgeranyl pyrophosphate (GGPP).

Statin induced myalgia and muscular toxicity syndrome are gaining more and more attention especially in older patients with sarcopenia.

Statin administration could contribute to an enhanced risk of sarcopenia in older people. Sarcopenia is considered a geriatric syndrome characterized by a progressive decline in skeletal muscle mass, muscle strength and performance. It is thought to affect approximately 10% of people aged 60-70 years, and its prevalence increases with age reaching up to 50% in people over 80 years. There is evidence that the decline in the overall performance of skeletal muscle contributes to the loss of independence and frailty and increases the risk of falls. The presence of sarcopenia has been associated with an increased risk of incident disability and mortality.

Further common side effects of statins are myalgia and exercise induced myalgia, muscle weakness and/or rhabdomyolysis with or without creatine kinase elevation. The statin induced muscular toxicity syndrome is more prevalent than previously thought, reaching up to 29%.

Myalgia is a potential adverse effect of statin treatment. Myalgia is the medical term for muscle pain. Myalgia may be described as a muscular ache, heaviness, stiffness or cramping sensation. Pain may be associated with ligaments, tendons, and the soft tissues that connect bones, organs, and muscles. Myalgia can be acute (short-term) or chronic (long-term). Chronic myalgia is often the main symptom of muscle and bone conditions, as well as autoimmune diseases.

Myalgia, with or without muscle weakness, is the most common adverse effect associated with statin use and is reported to occur in up to 10% of people prescribed statins. Statin-associated myalgia is characterized by the symmetrical involvement of large and proximal muscle groups, in particular the legs. Symptoms typically begin within six months of initiating the statin.

Risk factors for statin-associated myalgia include, for example, frail, elderly females; people with co-morbidities such as chronic kidney disease and diabetes; people taking high doses of statins or medicines which interact with statins; and people with a genetic pre-disposition.

Statin induced myalgia represents a limiting factor for physical activity and thus potentially contributing to an increased risk for sarcopenia.

When myalgia is associated with muscle inflammation, this is referred to as myositis. Myositis is usually accompanied by an elevation in serum creatine kinase. Rhabdomyolysis occurs when the inflammation is associated with muscle fiber break-down, releasing myoglobin into the bloodstream.

Rhabdomyolysis is the breakdown of muscle tissue that leads to the release of muscle fiber contents into the blood. These substances are harmful to the kidney and often cause kidney damage. Serious complications of muscle cell damage are more likely to occur in patients with risk factors, such as co-morbid illness, dehydration or pre-existing renal impairment. These complications include acute kidney injury (AKI) and widespread vascular coagulation. Death due to rhabdomyolysis in patients taking statins is extremely rare. The distinction between myositis and rhabdomyolysis is not always clear, however, a broadly accepted criterion for rhabdomyolysis is serum creatine kinase levels more than ten times the upper limit of normal, with evidence of myoglobinaemia.

Cholesterol plays an important role in maintaining cell membrane function. Disruptions to cholesterol synthesis may affect membrane ion channels and thereby modify muscle membrane excitability. The levels of mevalonate, which is a precursor of cholesterol as well as of co-enzyme Q10 (ubiquinone), directly affect the levels of both cholesterol and ubiquinone. Reductions in mevalonate, resulting in reduction in CoQ10, have been suggested to interfere with cellular respiration and result in muscle toxicity.

Statins may cause a reduction in other synthetic precursors of cholesterol, which have functional roles in cellular protein physiology. Programmed cell death (apoptosis) in skeletal muscle may be triggered by statins. Statins have been shown to decrease muscle strength by altering the movement of calcium within animal muscle cells.

Statin toxicity also depends on factors involved in drug metabolism such as cytochrome P450s and CYP3A4 subfamily enzymes. Statins are also suspected of being involved or implicated in non-muscle adverse events, such as diabetes mellitus, cancer, cognitive impairment, liver damage and more.

Some embodiments of the present disclosure relate to a method for treatment, prevention or amelioration of a disease, syndrome, disorder or condition associated with statin-based therapy, the method comprising administrating a therapeutically effective amount of mevalonolactone to a subject in need thereof.

Current treatment of SAMS in mild cases consists solely of discontinuation of statin therapy alone, with possible rechallenge after resolution of symptoms; treatment of rhabdomyolysis is mostly supportive, and treatment of IMNM may include corticosteroids, methotrexate, IVIGs and rituximab, to variable levels of success. Since many medical facilities lack the ability to detect anti-HMGCR antibodies, and for the lack of better treatment modalities, seronegative patients may be treated with immunosuppression. In light of the present disclosure, mevalonolactone may offer a safe and effective treatment in cases of SAMS that persist after statin cessation, as well as cases of severe SAMS including rhabdomyolysis, and perhaps even IMNM.

None limiting diseases, syndromes, disorders or conditions which may treated with any one or more of the methods disclosed herein include myopathy, statin-associated muscle symptom (SAMS). immune-mediated necrotizing myopathy (IMNM), rhabdomyolysis, muscular toxicity syndrome, myalgia, sarcopenia, limb girdle muscular dystrophy (LGMD) or biallelic HGMCR mutation limb girdle muscular dystrophy (HGMCR-LGMD).

In some embodiments, the disease, syndrome, disorder or conditions is at least one of myopathy, SAMS and SAMS persists after statin cessation.

Embodiments of the present disclosure pertain to oral administration of mevalolactone.

In some embodiments, the isomer D-mevalolactone produced via biologic processes is employed. DL-Mevalonolactone is a molecule of the formula:

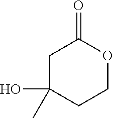

The term “oral administration” as referred to herein, is a route of administration where a substance is taken through the mouth. Per os abbreviated to P.O. is sometimes used as a direction for medication to be taken orally. Many medications are taken orally because they are intended to have a systemic effect, reaching different parts of the body, for example, via the bloodstream.

The active agent mevalolactone may be formulated in solid, liquid or semi-solid dosage forms. Dosage forms suitable for oral administration include, but are not limited to, pills, tablets (e.g., buccal, sublingual or chewable tablets), capsules, powders, pastilles, lozenges, granules, liquid solutions or suspensions (e.g., drinks, elixirs, syrups, oral drops), pastes, buccal films, and oils. Buccal are dissolved inside the cheek; sublingual dissolved under the tongue; tablets may be swallowed, chewed or dissolved in water or under the tongue.

Further oral dosage forms include sustained-release tablets and capsules (which release the medication gradually, powders or granules, and dops.

The term “therapeutically effective amount” as used herein, means the amount or dose of a compound, e.g., mevalolactone, that, when administered to a subject for treating a disease, disorder or condition, as defined herein, is sufficient to effect such treatment for the disease, disorder or condition. The therapeutically effective amount may sometimes be the lowest dose level that yields a therapeutic benefit to patients, on average, or to a given percentage of patients. The ‘therapeutically effective amount’ can vary depending on the compound, the disease and its severity, and the age, weight, etc., of the subject to be treated.

In some embodiments, a therapeutically effective amount is a dose of from about 1 mg/kg to about 200 mg/kg, D-mevalolactone, for example, from about 1 mg/kg to about 150 mg/kg, from about 1 mg/kg to about 100 mg/kg, from about 2 mg/kg to about 100 mg/kg, from about 3 mg/kg to about 150 mg/kg, from about 2 mg/kg to about 100 mg/kg, from about 1 mg/kg to about 5 mg/kg, from about 2 mg/kg to about 10 mg/kg, from about 5 mg/kg to about 20 mg/kg, from about 15 mg/kg to about 35 mg/kg, from about 10 mg/kg to about 40 mg/kg, from about 20 mg/kg to about 80 mg/kg, from about 30 mg/kg to about 90 mg/kg, from about 25 mg/kg to about 60 mg/kg, from about 50 mg/kg to about 200 mg/kg and any subranges and individual values therebetween.

In some embodiments, the dose is from about 8 mg/kg to about 50 mg/kg, from about mg/kg to about 30 mg/kg, from about 14 mg/kg to about 20 30 mg/kg, or about 16 mg/kg.

Any of the above-indicated doses may be provided to a patient from 1 to 5 times a week, for example, from 1 to 3 times a week.

The terms “therapy”, “treatment”, “treating”, “treat” as used herein are interchangeable and refer to: (a) preventing a disease, disorder, or condition from occurring in a human which may be predisposed to the disease, disorder and/or condition but has not yet been diagnosed as having it; (b) inhibiting the disease, disorder, or condition, i.e., arresting its development; (c) relieving, alleviating or ameliorating the disease, disorder, or condition, i.e., causing regression of the disease disorder and/or condition; and (d) curing the disease, disorder, or condition. In other words, the terms “therapy”, “treat,” “treatment,” and “treating,” extend to prophylaxis, namely, “prevent,” “prevention,” and “preventing,” as well as treatment per se of established conditions. Accordingly, use of the terms “prevent,” “prevention,” and “preventing,” would be an administration of the active agent to a person who has in the past suffered from the aforementioned conditions, such as, for example, myopathy or SAMS, but is not suffering from the conditions at the moment of the composition's administration.

Thus, the terms “treatment”, “therapy” and the like include, but are not limited to, changes in the recipient's status. The changes can be either subjective or objective and can relate to features such as symptoms or signs of the disease, disorder, syndrome or condition being treated. For example, if the patient notes relief in muscle pain, freedom to move and be physically active, then successful treatment has occurred. Similarly, if the clinician notes objective changes, such as by neurologic assessment, then treatment has also been successful. Preventing the deterioration of a recipient's status is also included by the term. Therapeutic benefit includes any of several subjective or objective factors indicating a desirable response of the condition being treated as discussed herein

### Process for Production of D-Mevalonolactone

The present inventors improved known method for the biological production of D-mevalonolactone. The improved process provided a yield at least 4 times higher that production yields obtained previously.

The biological process comprises the use of bacterial fermentation in a bioreactor and is described in Example 1 herein. The principal steps of the process include:
(i) Obtaining E. coli bacteria transfected with a plasmid containing the gene coding for the HGMCR enzyme, using any of the well-known transfection methods described in the art. Control of an inducible promotor (such as the Lac operon system) was employed. The transfected cells and are grown in Luria broth with appropriate antibiotic selection. Overexpressing genes that enhance HGMCR abundance may optionally be used to increase product yield.
(ii) Transferring the transformed bacterial cells are into a large volume of Terrific broth (TB) medium in a bioreactor system. The TB medium is a nutritionally rich medium for the growth of bacteria. The formulation of TB medium contains increased concentrations of peptone, yeast extract, and glycerol as a carbon source. In the process disclosed herein, TB medium is supplemented with appropriate antibiotic, antifoam and osmotic stress controlling additives or agents such as sorbitol and betaine. The addition of osmotic stress controlling agents to the fermentation medium is a modification of known biological fermentation process conducted in bioreactors, and this modification substantially improves the yield and affords at least 4 time as much product compared to corresponding process which do not employ agents that compensate for osmotic stress. The temperature, pH, levels of dissolved oxygen and glucose concentration are controlled during the fermentation process.
(iii) Growing the transformed bacteria cells until a sufficient biomass is achieved.
(iv) Inducing the transformed bacteria cells to synthesize D-mevalonolactone by the addition of an inducer such as isopropyl B-d-1-thiogalactopyranoside (IPTG)). Fermentation is maintained throughout D-mevalonolactone production at appropriate pH, dissolved oxygen, temperature, and levels of nutrients, by glucose monitoring, pH-stat feeding and the like.
(v) Separating the bacterial cells from the broth medium that contains the synthesized product. After fermentation ceases, the cell culture broth is centrifuged once to separate the cells and the pelleted cells are discarded.
(vi) Extracting the D-mevalonolactone via organic extraction, which is facilitated by the following steps: (a) acidification to a pH 2 to allow conversion of mevalonate to mevalonolactone; and (b) addition of salt to saturation level in order to “salt-out” and remove remaining cells and proteins. After acidification and salting out, the solution is clarified by centrifugation or filtration to remove insoluble remains.

Organic extraction is done using ethyl acetate or other appropriate organic solvents. The organic phase is maintained and evaporated.

Additional organic extraction, acid-base extraction, distillation, column chromatography and other purification methods can be used to increase purity.

The terms “comprises”, “comprising”, “includes”, “including”, “having” and their conjugates mean “including but not limited to”.

The term “consisting of” means “including and limited to”

As used herein, the singular form “a”, “an” and “the” include plural references unless the context clearly dictates otherwise. For example, the term “a compound” or “at least one compound” may include a plurality of compounds, including mixtures thereof.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Various embodiments and aspects as delineated hereinabove and as claimed in the claims section below find experimental support in the following examples.

### EXAMPLES

Reference is now made to the following examples, which together with the above descriptions illustrate some embodiments in a non-limiting fashion.

### Materials and Methods

### Patients

Patients that participated in the clinical study were afflicted with a muscular disorder in an apparent autosomal recessive mode-of-inheritance, which was discovered and determined by the present inventors as a new form of genetic LGMD caused by mutated HMGCR.

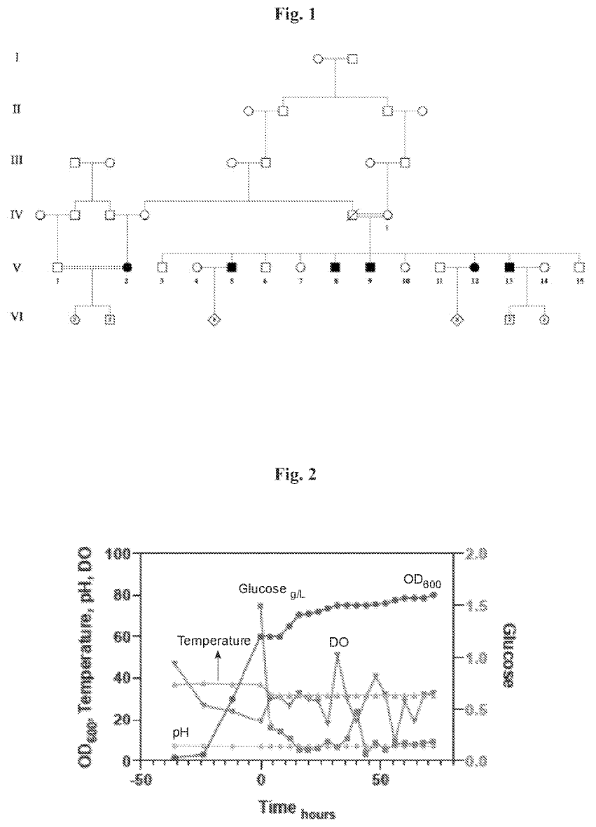

Six individuals of a single consanguineous Bedouin kindred (see FIG. 1) were affected with the same disease. The patients developed limb-girdle muscular dystrophy, initially manifesting as muscle fatigue, mainly of the proximal muscles, with an onset during the 4th decade of life. Muscle symptoms were associated with pain on exertion at an earlier stage, progressively affecting mostly the proximal and axial muscles, culminating with involvement of respiratory muscles. The phenotype was non-remittent and progressive; the oldest three patients (V: 2, 5 and 12 ages 49, 58 and 51, respectively) were unable to walk, were wheelchair-bound or bedridden, and suffered from respiratory insufficiency; patient V: 2 was chronically ventilated by tracheostomy. Distal and facial muscles were mostly spared in all patients, and none suffered from any other neurological deficits or dysphagia.

Imaging studies of the CNS showed no pathological process in all patients. MRI, CT and ultrasound scans showed various levels of atrophy with severe fatty replacement of skeletal muscles of large proximal muscles and axial muscles with sparing of the distal and facial muscles. These radiological features were distinct from known forms of limb girdle muscular dystrophy and were highly reminiscent of the MRI features of statin myopathy and immune-mediated necrotizing myopathy (IMNM). Electromyography (EMG) studies of four patients showed a myopathic pattern, and nerve conduction study (NCS) demonstrated normal distal latencies, amplitudes, and velocities. There were also no clear signs of cardiomyopathy; cardiac and chest CT scans done for patients V: 9 and V: 5 were normal, and echocardiography in 5 patients showed no abnormalities. Abdominal ultrasound demonstrated no pathological features.

Pathological evaluation of muscle biopsies taken from the biceps or deltoid muscles of three patients, did not reveal any pathological features including fibrosis, inflammation, or any other abnormalities. Evaluation of thin sections by electron microscopy was normal as well.

Laboratory investigations showed muscle injury with elevated creatine kinase (CK), an enzyme found in skeletal muscle, heart muscle and brain. When any of these tissues are damaged, they leak creatine kinase into the bloodstream. Elevated CK levels may indicate muscle injury or disease. Up to over X 250 upper level of normal CK (maximal CK of 35761U/L in patient V: 8) was detected and elevated transaminases in all patients, and occasionally a mild rise in alkaline phosphatase which was seen only for the patients with severe phenotype. A rise in alkaline phosphatase is not uncommon, while the rise in CK is dramatic in comparison to other forms of muscular dystrophies. With disease progression and muscular atrophy, CK levels gradually normalized and eventually even dropped beneath normal limits, with simultaneous drop in creatinine levels, indicating low skeletal muscle mass. The patients did not display elevated levels of cholesterol and lipoproteins, with most patients frequently showing total cholesterol levels lower than 120 mg/dL. All patients had elevated fasting blood sugar levels above 126 mg/dL, with patient V: 13 treated with insulin. Other than muscle symptoms and high blood sugar levels, no other overt pathologies were shared between the patients.

Genetic evaluation of the patients included whole-exome sequencing of patient V: 13 and patient V: 2, and whole-genome SNP genotyping for all available affected and non-affected family members were analyzed as previously described (Wormser et al., Eur J Hum Genet, 2019, 27 (6): 928-40. Available from http://www.nature.com/articles/s41431-019-0347-z; Seelow et al., Nucleic Acids Res., 2016, 26:37. Available from https://academic.oup.com/nar/article-lookup/doi/10.1093/nar/gkp369). Genome-wide linkage analysis identified a single 3.2Mbp homozygous segment that was shared among all affected individuals and was either absent or found in a heterozygous state in unaffected individuals. Linkage analysis (multipoint and two-point) of chromosome 5 showing the shared locus and logarithm of the odds (LOD) score calculation were performed. An LOD score is a statistical estimate of the relative probability that two loci (e.g., a disease-associated gene and another sequence of interest, such as a variant or another gene) are located near each other on a chromosome and are therefore likely to be inherited together. The disease-associated locus was found on chromosome 5q13.2-q13.3, spanning between SNPs rs2129403 and rs2914143, 5:73803333-77084175 (GRCh38/hg38), and showed a maximal LOD score of 4.8204 at rs4345300.

Following exome data filtering, only a single variant was found within the 3.2 Mbp locus. The mutated nucleotide coded amino acid, and the entire gene sequence was highly conserved throughout evolution; the Glycine to Aspartate substitution at position 822 was a radical replacement, predicted to interfere with several peptide bonds, disrupt helix-dipole and cause charge-based repulsion, with possible detrimental impact on secondary and tertiary structure of HMG-COA reductase.

Within the locus, there were no other variants, nor were there any variants in genes known to cause LGMD and LGMD-like diseases throughout the exomes. The HMGCR variant was validated via restriction analysis and Sanger sequencing (data not shown) and was found to fully segregate as expected for autosomal recessive heredity in the studied kindred. Of 190 non-related ethnically matched controls of tribes other than the one affected, none carried the variant. Screening of 20 individuals of the same tribe did not show other carriers.

Six affected individuals ages 37-58 of a single consanguineous Bedouin, kindred from the Israeli Negev region were studied (FIG. 1). Blood samples for DNA, RNA and various other tests were obtained from patients and unaffected family members following Soroka University Medical Center (SUMC) IRB approval and informed consent. Clinical phenotyping was determined by experienced neurologists and geneticist for all affected individuals, and genetic counseling was offered to direct family members. Imaging studies, electromyography (EMG), nerve conduction velocity (NCV) studies and muscle biopsies were conducted in accordance with common standards of medical practice and using common techniques.

### Animals

All animal studies were conducted under the approval of institutional animal care and use committee (IACUC) and in accordance with standards of animal care. Mice (C57B6/J) were purchased from Harlen, Israel.

### Protein Purification

HMGCR transcript NM_000859.3 was cloned from human skeletal muscle cDNA (Takara™, Shiga, Japan) into pJET vector using CloneJET PCR cloning kit (Thermo Scientific™, MA, USA). Mutagenesis was performed using standard technique. The catalytic portion of HMGCR (p.426-888) was sub-cloned into pGEX-6p plasmid (Cytiva™, MA, USA). Protein expression and purification were performed as described by Istvan et al. (Istvan et al., EMBO J., 2000, 19 (5): 819-30. Available from http://www.ncbi.nlm.nih.gov/pubmed/10698924) with minor modifications. Briefly, pGEX-6P-HMGCR-426-888 was transformed to E. coli BL21 cells. Cells were grown in 2×YT broth (for growth of hosts for replication vectors, comprising NaCl, Peptone 140 (pancreatic digest of casein, and yeast extract) with 0.5 M sorbitol and 2.5 mM betaine and 100 mg/L ampicillin in 6 L batches until OD600=0.6, after which expression was induced with 0.5 M isopropyl B-d-1-thiogalactopyranoside (IPTG) and collected after 16 hr. Cell pellets were resuspended in Buffer A (bacteria lysis buffer, comprising 20 mM K₂HPO₄ (pH=7.4), 20 mM Tris (pH=7.6), 500 mM (NH₄) 2SO₄, 1 mM EDTA, 2 mM TCEP, 1 mM MgCl₂, 10% glycerol, 0.01% and Triton X-100) disrupted using a FRENCH® Press (Thermo), and cleared lysates were purified by affinity chromatography using Pierce™ Glutathione Agarose (Thermo), washed with Buffer B (wash buffer and elution buffer, comprising 20 mM K₂HPO₄, 20 mM Tris (pH=8), 200 mM (NH₄)2SO₄, 1 mM EDTA, 2 mM TCEP, 10% glycerol). For elution of GST-fused protein 10 mM reduced glutathione was added) and either eluted with Buffer B with added glutathione, or washed with Buffer E (HMGCR protein and assay buffer, comprising 100 mM K₂HPO₄, 120 mM KCl, 1 mM EDTA, 2 mM TCEP) digested on-column with HRV3C protease (SAE0045, Sigma) and then eluted with Euffer E. Protein was further purified using ÄKTAprime plus (Cytiva) system, using size exclusion on a SuperDex200 column, followed by ion-exchange using a Hitrap Q HP column with Buffers C and D (Buffer C is ion-exchange low salt buffer, comprising 50 mM K₂HPO₄, 50 mM KCl, 1 mM EDTA, 2 mM TCEP. Buffer D is ion-exchange high salt buffer, comprising 50 mM K₂HPO₄, 1 M KCl, 1 mM EDTA, 2 mM TCEP). Lastly, purified protein was dialyzed and concentrated using Amicon® column (Merck-Millipore, MA, USA) and Buffer E and aliquots of purified protein in Buffer E were flash frozen at a concentration of 0.6 mg/mL. The purified protein was analyzed by western blot with a rabbit monoclonal anti-HMGCR antibody (SAB4200528, Sigma, MO, USA).

### HMG CoA Reductase Catalytic Assay

HMG CoA reductase enzymatic activity was assessed by a colorimetric assay. HMG CoA reductase reactions were set up in 96 well plates, with each well containing a total volume of 100 μL Buffer B, supplemented with 400 μM nicotinamide adenine dinucleotide phosphate (NADPH) (Sigma) and 2 μL of either: (i) wild type (WT) purified HMG CoA reductase protein; (ii) mutant HMG CoA reductase protein; (iii) WT enzyme with pravastatin (see further information below); (iv) or a no enzyme (control). Reactions were initiated by the addition of HMG-COA substrate to a final concentration of 0-400 μM. Immediately after addition of HMG-COA, plates were analyzed for 340 nm absorbance using an Infinite M200 plate reader (Tecan, Mannedorf, Switzerland). Absorbance was monitored every 30 seconds for a total of 90 minutes. While repeating measurements, HMG CoA was added to either WT or mutant reactions in an alternating fashion in order to minimize technical variations in Vo.

### Isothermal Calorimetric (ITC) Analysis

Isothermal calorimetric studies were performed on a nano ITC instrument (TA Instruments, DE, USA). In order to facilitate pure substrate binding measurements and to avoid the reaction generated by HMG CoA reduction to mevalonate, the thermodynamic of HMGCR binding with pravastatin were analyzed (Carbonell et al., Biochemistry, 2005, 44 (35): 11741-8. Available from http://pubs.acs.org/doi/pdf/10.1021/bi050905v). HMG CoA reductase WT and mutant protein at a concentration of 20 μM in Buffer B supplemented with 1 mM NADPH in the reaction well were injected with 50 UM pravastatin in Buffer B supplemented with 1 mM NADPH using the multiple injection mode for a total 20 7 μL injections. Heat data were plotted using Nano Analyze software™.

### Anti-HMGCR Antibodies Assay

Serological examination of patient serum for anti-HMGCR autoantibodies was performed as previously described (Mammen et al., Arthritis Rheum, 2018, 63 (3): 713-21. Available from http://www.ncbi.nlm.nih.gov/pubmed/21360500). Briefly, Nunc MaxiSorp™ plates were coated with 100 ng of WT HMGCR protein overnight at 4° C. Diluted serum from patients and controls (1:200) was added to the plate and incubated 1 hr at 37° C. Plates were washed and incubated with a rabbit anti-human-HRP antibody (ab5679, Abcam, Cambridge, UK). Plates were developed using Pierce™ TMB Substrate Kit (Thermo) and analyzed with an Infinite M200 plate reader (Tecan). Rabbit anti-HMGCR antibody 1:5,000 and phosphate buffer saline (PBS) were used as positive and negative controls, respectively.

### D-Mevalonolactone Liquid Chromatography-Mass Spectrometry (LC-MS) Analysis

Serum samples from patients and healthy volunteers were assessed by LC-MS. Sample preparation and chromatographic analysis were based on a previously described method (Cestari rt al., J. Pharm Biomed Anal., 2020, 182:1-7. Available from https://linkinghub.elsevier.com/retrieve/pii/S0731708519327141). Briefly, 100 μL serum samples or mevalonolactone standard (25-0 ng/ml in an 8-point serial dilution with high pressure liquid chromatography (HPLC)—grade water) were added to 200 μL 0.1 M HCl and were shaken for 30 minutes at room temperature. Organic extraction was performed by addition of 1 mL ethyl acetate. Samples were shaken at room temperature for 1 hr, centrifuged at 3,000 g for 10 minutes, and 800 μl were transferred to a new HPLC vial and evaporated to dryness using a Savant SpeedVac™ (Thermo). Lastly, samples were reconstituted in 100 μl of 80% methanol, 20% water and 0.1% formic acid. Sample injections (10 μL) were separated on an Acquity HSS-T3 1.8 μm, 2.1×100 mm column and were analyzed in a Q Exactive focus hybrid quadrupole LC-MS system. Spectra were analyzed using the FreeStyle™ software.

### Statistical Analysis

All results were analyzed using GraphPad Prism V9 with student t-test, ANOVA analysis, and Michaelis-Menten kinetics studies. Data from PhenoMaster was also evaluated using CalR software.

### Example 1

### Bioreactor Synthesis of Mevalonolactone

### (i) Bioreactor Process

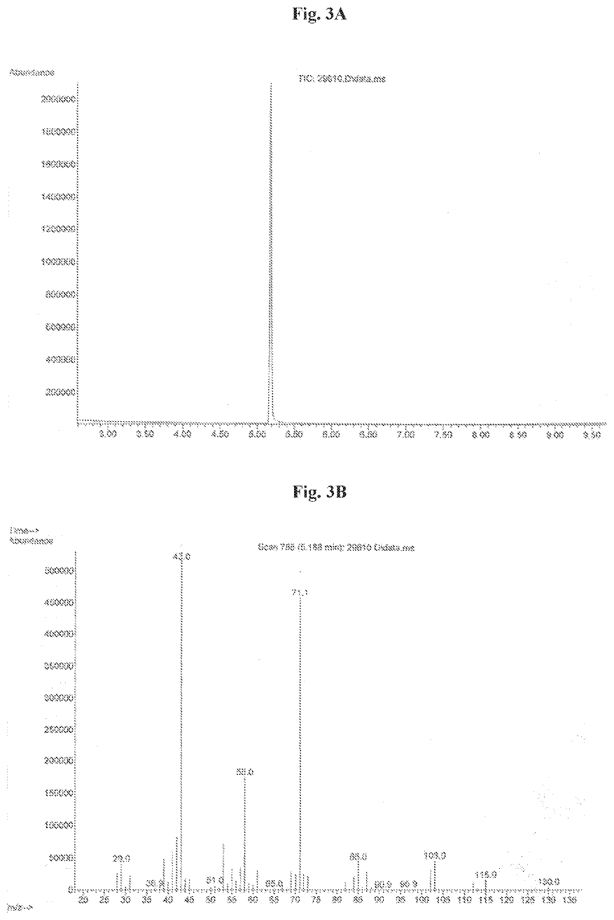

D-Mevalonolactone synthesis was performed using bacterial fermentation in a bioreactor, as follows: DH10B-strain E. coli cells, harboring the pMevT plasmid, were cultured overnight at 37° C., 180 RPM in a 250 mL shaker flask containing 100 ml of Luria broth (LB) media supplemented with 30 μg/mL chloramphenicol. Luria broth is a rich medium that is commonly used to culture members of the Enterobacteriaceae as well as for coliphage plaque assays. LB and related media (e.g., 2×YT, Terrific broth (TB)) are used extensively in recombinant DNA work and other molecular biology procedures. The culture was then used to inoculate a 10 L benchtop Jupiter bioreactor (Solaris, Porto Mantovano, Italy), containing 6 L TB (20 g/L tryptone, 24 g/L yeast extract, 4 ml/L glycerol) media supplemented with 30 μg/mL chloramphenicol, 0.01% antifoam, 0.5M sorbitol and 25 mM betaine. The bioreactor was maintained at 37° C. until the biomass reached OD600=30-60, after which the culture was induced using 1 mM isopropyl β-d-1-thiogalactopyranoside (IPTG) and the temperature was reduced to 32° C. and maintained for the entire duration of fermentation. During fermentation, the pH was set at 7.0±0.1 by automatic addition of 25% ammonium hydroxide. Dissolved oxygen (DO) was maintained at 30% by a cascade function adjusting stirring rate, air flow and lastly O₂ flow, and the culture was fed with 40% glucose solution. Glucose concentration was kept under 0.5 g/L (50 mg/dL) using either manual tuning of glucose feeding speed or by pH-stat feeding by a logic loop operating such that whenever pH raised above 7, glucose flow was initiated at a rate of 0.1 mL/min, and when pH drops below 7, glucose flow was stopped. Antifoam was added automatically. DO, pH and temp were monitored off-site using the Solaris software and were also monitored and adjusted every 4 hours, when manually measuring OD600 and glucose levels. Fermentation batches were cultured for 16-120 hr. Additional chloramphenicol and IPTG were added every 12 hours. An exemplary time course of mevalonolactone batch fermentation process is shown in FIG. 2.

### (ii) Extraction and Purification of D-Mevalonolactone

Cell culture broth was centrifuged at 12,000 g, 4° C. for 10 minutes, cell pellets were discarded, and the remaining broth was acidified to pH 2 using 16% HCl and incubated at 45° C. to facilitate conversion of mevalonic acid to mevalonolactone. The solution was saturated with NaCl and the resulting protein precipitates were discarded after either filtration using standard filter paper, centrifugation at 12,000 g, 4° C., for 10 minutes or both. The broth was then cooled and extracted for a minimum of 3 times using ethyl acetate in a 1 L separatory funnel. Hard emulsions were either discarded or centrifuged at 3000 g for 5 minutes in glass tubes, and phases were separated and collected. The organic phase was evaporated using a rotary evaporator, and the remaining mevalonolactone was placed under high vacuum (10⁻³ torr) overnight.

Batches failing to show >94% purity by GC-MS were further purified by silica column chromatography, using ethyl acetate alone as the mobile phase. GC-MS analysis was used to inspect the resulting fractions. Other samples were further purified using acid-base extraction: samples were dissolved in ultra-pure water; pH was adjusted to 10 and the solution was extracted 3 times using ethyl acetate to remove all non-acidic components. The resulting solution was adjusted to pH 2 and extracted 3 times with ethyl acetate. The organic phase was then dried as described.

(iii) D-Mevalonolactone Purity Analysis

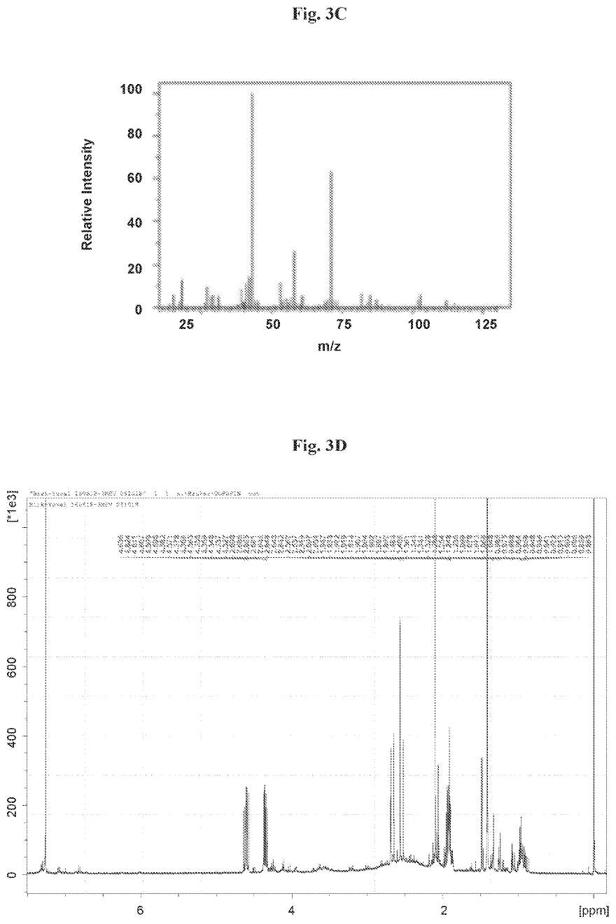

Purity was inspected by gas chromatography-mass spectrometry (GC-MS) and nuclear magnetic resonance (NMR). Samples were dissolved in ethyl acetate and subjected to GC-MS on an Agilent 5977A GC/MSD system: 7890B Agilent GC system with an ultra-inert GC column (19091S-433UI) followed by an Agilent 5977A MS instrument. Helium was used as the carrier gas at a constant flow of 0.7 mL/min, and 1:50 split samples were injected. Injection port and MS source temperature were held constant at 230° C., and the MS quad temperature was held constant at 150° C. The column temperature gradient was as follows: 70° C. for 0.5 min, 25° C./min to 150° C., 15° C./min to 200° C., 25° C./min to 300° C. and held at the upper temperature for 1 min. Results were analyzed on Mass Hunter software, Mass spectrum was compared to Wiley/NIST 2014 library. For NMR analysis, samples were dissolved in deuterated chloroform (CDCl₃) in NMR tubes and were analyzed using an AVANCE III-400 device (Bruker) in 1H, 13C and DEPT NMR modes. Resulting spectra were compared to AIST spectral database. The purity analysis results shown in FIGS. 3A-3E indicate the obtention of pure mevalonolactone.

### Example 2

### D-Mevalonolactone Toxicity Study

All animal studies were conducted under the approval of institutional animal care and use committee (IACUC) and in accordance with standards of animal care. The safety of oral treatment with mevalonolactone was assessed by oral gavage. Mevalonolactone administered was produced by batch-fermentation as described in Example 1 above and was purified to over 94%. Impurities were analyzed and were all ascertained as non-hazardous. Gavage was performed using a 22-gauge olive tip curved gavage needle coated with a sugar solution, with 3 doses of 20, 200 and 2000 mg/kg of mevalonolactone or 0.9% saline solution daily for 7 days, to assess toxicity and chemical damage potential (N=5 for each group). Dose of 2000 mg/kg/day is more than 5 times the maximal weekly dose intended for human use. Blood samples were collected at day 0 from the mandibular vein. At day 7, mice were sacrificed, blood samples were collected, and organs were fixed with formalin, embedded in paraffin, sectioned, and stained with (i) hematoxylin and eosin (H&E) staining (hematoxylin stains cell nuclei a purplish blue, and eosin stains the extracellular matrix and cytoplasm pink, with other structures taking on different shades, hues, and combinations of these colors); (ii) and periodic acid-Schiff (PAS; for detecting polysaccharides such as glycogen, and mucosubstances such as glycoproteins, glycolipids and mucins in tissues). Blood, gastro-intestinal (GI) tract, liver and muscle samples were examined. Slides were reviewed by an expert veterinary pathologist (Patho-Logica LTD, Israel).

No major differences between groups were seen. Notably, high potassium levels were observed in all groups, including the untreated controls. This can be explained by the fact that blood studies for all groups was performed after CO₂ inhalation, which is known to increase potassium.

### Example 3

### Assessment of Mutated HMG-COA Reductase Biological Function

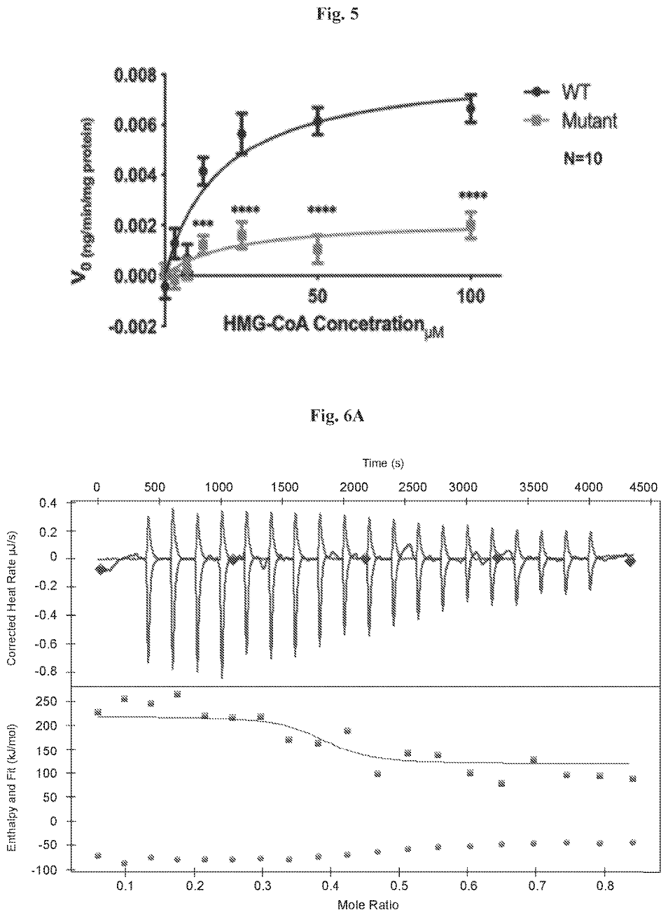

To validate the pathogenicity of the HMGCR mutation, functional analysis of the wildtype and mutant forms of the HMG-COA reductase protein (HMGCR) were performed. Efficacy of the enzyme to convert the HMG-COA substrate to mevalonic acid was evaluated. Spectro-colorimetric analysis of the WT and mutant proteins' function using an NADPH-oxidation assay was performed as described in Materials and Methods above. The results are shown in Table 1 and FIG. 5.

TABLE 1
HMG-CoA reductase (HMGCR) wild type and mutant activity

| | Wild Type | Mutant |
|---|---|---|
| Vₘₐₓ (ng/min/mg protein) | 7.28 ± 0.56 | 2.29 ± 0.49 |
| Kₘ (μM) | 13.73 ± 3.83 | 22.66 ± 8.27 |

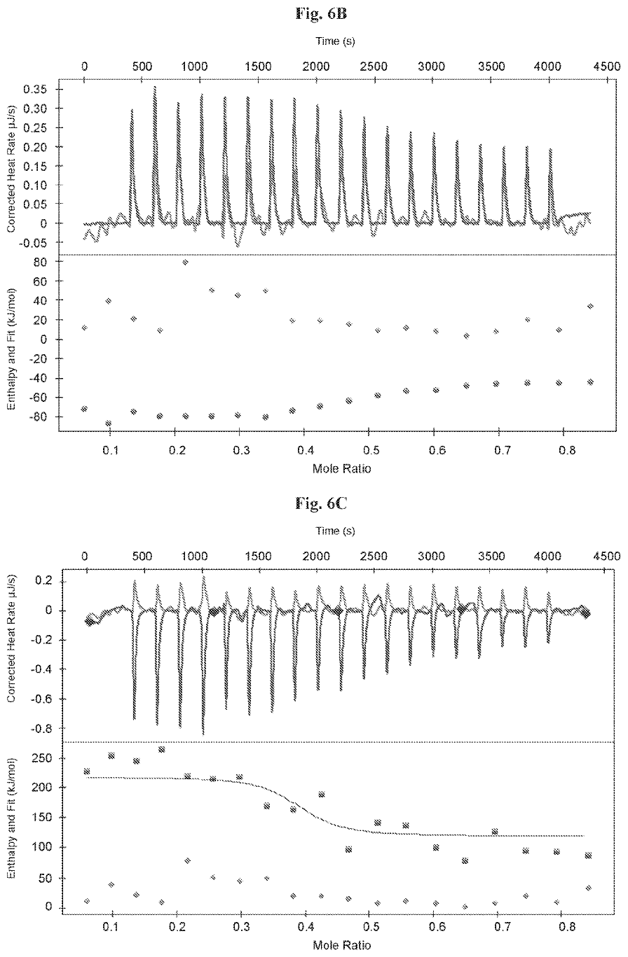

The mutant protein presented a 69% reduction in Vₘₐₓ and 65% increase in Kₘ in relation to the substrate HMG-COA, indicating lower affinity of the mutated protein for HMG-COA as well as overall slower reaction-rate (FIG. 5, Table 1)). This was also supported by isothermal calorimetric (ITC) analysis of the thermodynamics of HMG-COA reductase to a known inhibitor, pravastatin. The overall thermodynamic values of the ITC assay using HMG-COA as the analyte would represent both binding of the substrate to HMGCR, as well as the thermodynamics of the reduction reaction. To avoid this, pravastatin, a statin, which binds to the same catalytic pocket as HMG-COA, was selected. As seen in FIGS. 6A-6C, the WT protein showed a mild exothermic reaction, while the mutant form displayed kinetics almost identical to a no-protein control, indicating a very low affinity of the proteins' catalytic portion towards statins.

### Example 4

D-Mevalonolactone Treatment of Patient Afflicted with a Novel Form of LGMD Associated with HMGCR Mutation

Treatment with D-mevalonolactone was conducted under the approval and supervision of the drug committee safe use of medicines (SUMC) and the Israeli Ministry of Health (MOH) under an “expanded access/compassionate use” protocol, given the severe condition of patients and the lack of alternative treatments for LGMD associated with HMGCR mutation. The patient treated was patient V: 2 (see FIG. 1). Patient was instructed as to the risks and possible benefits and granted signed informed consent.

The treatment trial was monitored by a senior neurologist, and safety and efficacy reports were issued to the MOH in accordance with standard procedure.

Prior to initiation of treatment, the patient was thoroughly examined, including a neurological exam by a senior neurologist, imaging studies, electrophysiological studies, and blood work. Alongside severe limb girdle myopathy, a notable finding was the lack of Anti-HMGCR antibodies in all patients. No other rheumatological abnormalities were detected. Serum mevalonolactone level of patient V: 2 (determine as described in Materials and Methods) was 14.5-29.1% lower than the normal average.

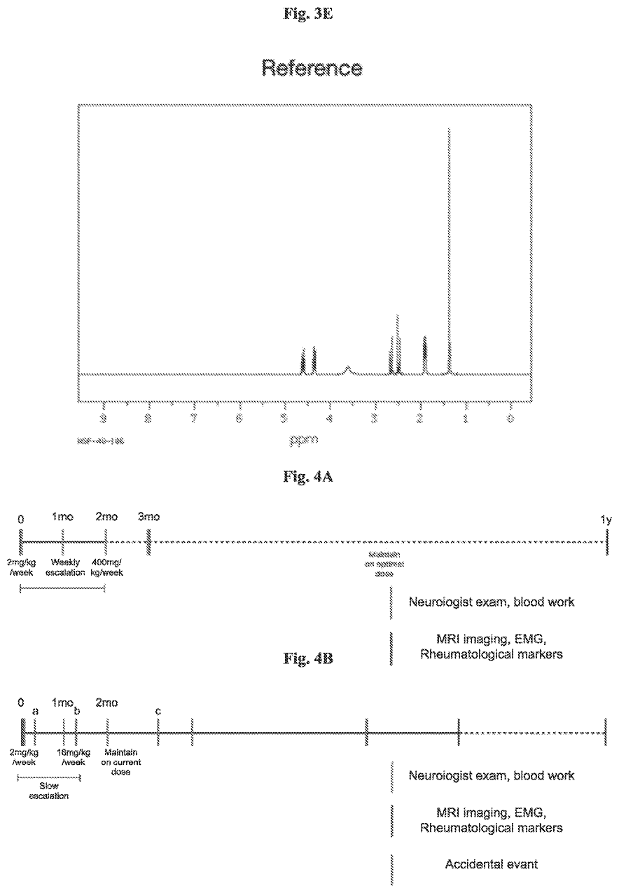

Treatment protocol was planned as an escalating dose of oral D-mevalonolactone dissolved in water or encapsulated in gelatin caps, given once weekly. The intended treatment plan is schematically shown in FIG. 4A. Practically, the treatment plan was modified to facilitate the patients' needs, coincidental events and therapeutic goals, as seen in FIG. 4B.

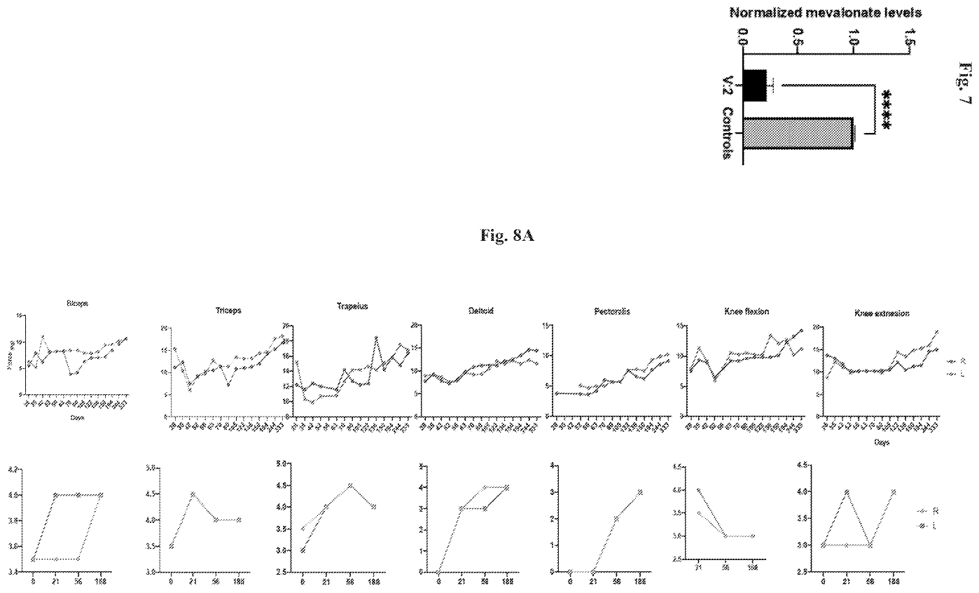

Initial treatment plan included a single weekly oral dose of mevalonolactone diluted in tap water starting at 2 mg/kg/week with a weekly doubling up to 400 mg/kg/week. However, since subjective and objective improvement was noticed, the dose was maintained at 16 mg/kg, given up to 3 times a week. Mevalonolactone oral uptake was found to be very rapid: mevalonate levels began to rise 20 minutes after oral administration, peaked at 50 minutes and returned to baseline levels 2 hours after ingestion (FIG. 7).

During treatment the patient was monitored closely, with weekly questioning, physical examination, spirometry, blood tests and manual muscle dynamometry (Lafayette instruments®, IN, USA) done by a member of the trial staff, and bi-monthly exam by a senior neurologist. Whole-body MRI, thorough blood investigation, echocardiography, abdominal ultrasound, electromyography (EMG) and nerve conduction study (NCS) tests were done prior to commencement of treatment and one year into the treatment. Electromyography measured muscle response or electrical activity in response to a nerve's stimulation of the muscle. Nerve conduction velocity measured how fast electrical signals moved through a nerve and was performed to evaluate nerve function. These tests assessed the muscles for abnormalities.

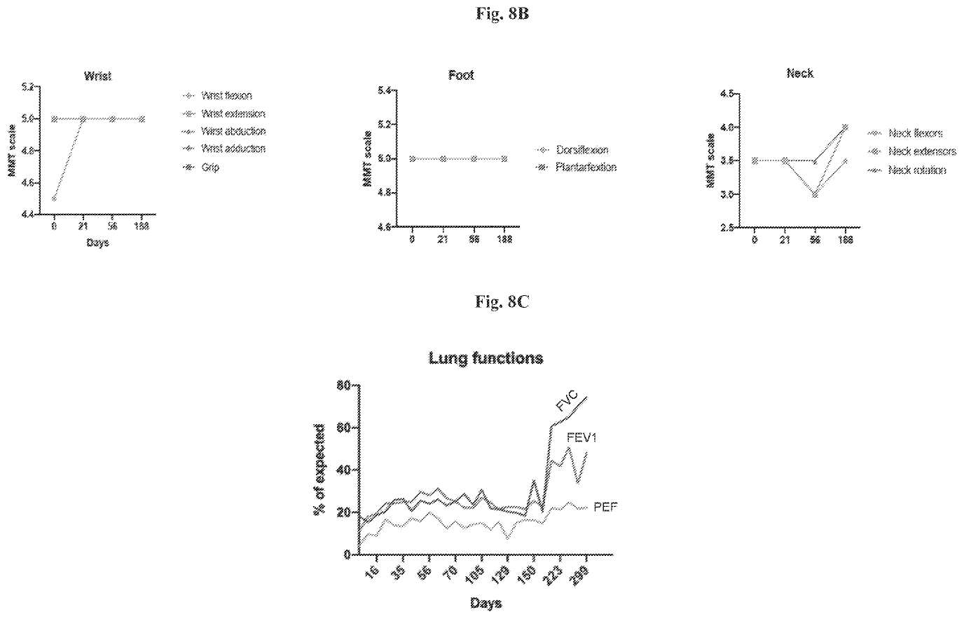

During treatment there was improvement in muscle strength as assessed by dynamometry and by manual muscle testing MMT (FIGS. 8A-8B). Improvement was noticed among all muscle groups, with the greatest improvement seen in the deltoid muscles. Distal muscles of the lower limb showed normal strength at the beginning of the trial and were not assessed by dynamometry after 3 months into the trial.

Respiratory muscles were assessed by spirometry, which demonstrated marked increase in peak expiratory flow, Forced vital capacity and FEV1 (FIG. 8C).

In addition to objective measurements of muscle strength, the patient showed remarkable improvement in independence and function in activities of daily living that were not feasible prior to treatment initiation. For example, the patient was able to raise herself from lying sideways to a sitting position, to fully abduct her arm when laying, to extend her knees to 150°, to feed her grandchild on her own and to stand with assistance. Her respiratory muscles also improved, and she was able to breath without her ventilator for over 2 hours while maintaining O₂ saturation >96%.

Adverse effects to the treatment were minimal. While throughout the one year treatment period there were occasional rare gastrointestinal symptoms, including mild nausea, diarrhea, constipation, urinary tract infections, a single occasion of near syncope, moderate headaches, rare temporary occurrences of a subjective sensation of limb swelling and of borderline mild dilatation of small veins in the upper and lower limbs, none of those episodes was unusual in frequency or severity compared to pre-treatment, and none of those minor episodes could be directly related to the treatment, required medical treatment or hospitalization. However, noticeable pigmentation of the proximal nail fold was seen on 4 different occasions in direct relation to treatment. The discoloration appeared shortly after treatment and vanished after 2-3 days of mevalonolactone ingestion and did not appear to be in relation to any vascular insufficiency, as assessed by presence of peripheral pulses, normal capillary refill, absence of pain and point-of-care doppler ultrasonography (Vscan extend, GE Healthcare, IL, USA). With the continuation of treatment, this effect subsided but did appear after dose escalation and after intermissions in treatment regimen.

### Example 5

### Mevalonolactone Treatment in a Model of Statin Myopathy

HMG-CoA reductase is the drug target of the lipid-lowering drug class statin, which is one of the most prescribed drug class in the world. Statins are well known for their most common adverse effect, statin-induced myopathy which may affect up to 30% of treated patients. Given the apparent safety of mevalonolactone oral treatment in HMGCR-LGMD, its utility in the treatment of a more prevalent condition, statin myopathy, was assessed employing a murine model of severe statin-induced myopathy (Chung et al., PLOS One, 2020, 11 (12): e0168065. Available from: https://dx.plos.org/10.1371/journal.pone.0168065; Meador and Huey, Muscle Nerve, 2021, 44 (6): 882-889. Available from: http://doi.wiley.com/10.1002/mus.22236).

C57B6/J mice were randomized to six treatment groups), n=4 for each group: (i) (ii) cerivastatin (ab142853, abcam) 1 mg/kg/day intraperitoneally (IP) injected. Cerivastatin was injected at a concentration of 0.3 mg/mL, injections volume was calculated by animal weight; (ii) Cerivastatin IP injected with 200 mg/kg/day mevalonolactone in provided in drinking water; (iii) simvastatin, alkaline hydrolyzed (sc-200829, Santa Cruz biotechnology, TX, USA) 20 mg/kg/day IP injected without mevalonolactone. Simvastatin was injected at a concentration of 5.4 mg/mL, injections volume was calculated by animal weight; (iv) Simvastatin IP injected with 200 mg/kg/day mevalonolactone in provided in drinking water; (v) sterile 0.9% saline solution, IP injected; and (vi) 0.9% saline solution IP injected with 200 mg/kg/day mevalonolactone in drinking water.

Treatment was initiated at age 8 weeks for a duration of 14 days. Mice were injected and monitored daily and weighed every 3 days. Assessment of muscle strength and endurance was performed using the hanging wire assay at day 0 and at days 4, 7, 11 and 14; a strength test using a grip strength meter 1027SM (Columbus Instruments, OH, USA) on day 14; and by measuring locomotor activity in a PhenoMaster home-cage phenotyping system (TSE systems, Germany) equipped with ActiMot3, and Voluntary Running Wheel modules from day 11 to 14. At the end of day 14 mice were sacrificed by inhalation of isoflurane followed by cervical dislocation and blood and organs were collected.

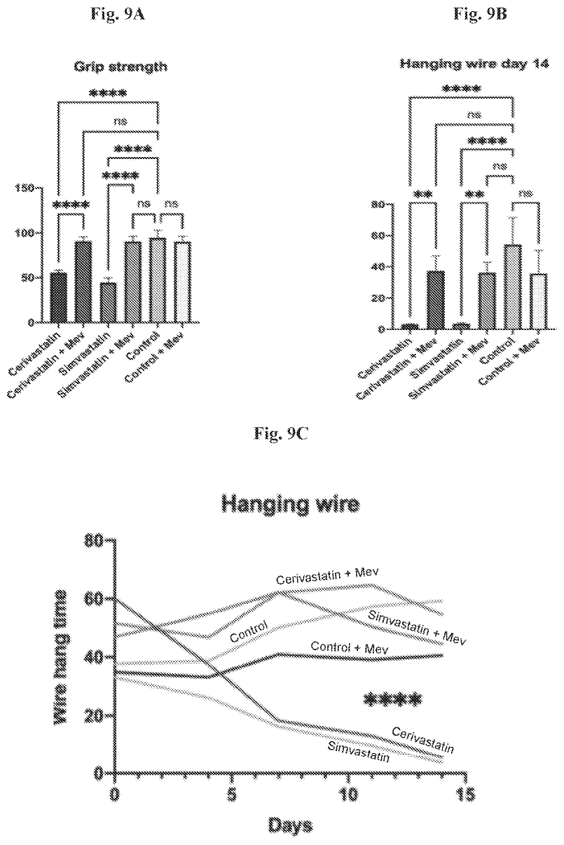

Mice given statins showed decreased muscle strength and endurance as assessed by wire hanging and grip force tests (FIGS. 9(A)-9(B)), and a lower tendency to initiate and pursue movement as observed in total ambulation and voluntary wheel activity and maximal speed. Importantly, animals that were treated with statins and given free access to mevalonolactone-supplemented drinking water showed a greatly reduced phenotype, most prominently observed in the hanging wire experiments (FIGS. 9(B)-9(C)). Muscle histology of mice treated with statins with or without mevalonolactone did not show overt signs of necrosis or inflammation. This is in line with the histopathological specimens of HMGCR-LGMD patients, which did not show gross abnormalities, and with histological findings in biopsy samples of statin-related myopathy patients, which often do not show pathological features.

The invention claimed is:

1. A method for treating, preventing or alleviating at least one of:
(i) impaired mevalonic acid production;
(ii) cell and/or tissue damage caused at least in part due to impaired mevalonate pathway; or
(iii) cell and/or tissue damage caused at least in part due to impaired mevalonic acid production, comprising administering a therapeutically effective amount of mevalonolactone to a subject in need thereof, thereby treating, preventing or alleviating the impaired mevalonic acid production or the cell and/or tissue damage in the subject.

2-3. (canceled)

4. The method of claim 1, wherein the damaged cell or tissue is muscle cell or muscle tissue.

5. A method for treating, ameliorating or preventing a disease disorder or condition associated with at least one of:
(i) impaired mevalonate pathway;
(ii) impaired mevalonic acid production; or
(iii) statin-based therapy,
comprising administrating to a subject in need thereof a therapeutically effective amount of mevalonolactone, thereby treating, ameliorating or preventing the disease disorder or condition in the subject.

6. (canceled)

7. The method of claim 1, wherein the impaired mevalonate pathway or the impaired mevalonic acid production is associated with impaired function of the enzyme 3-hydroxy-3-methyl-glutaryl-coenzyme A reductase (HMGCR).

8. A method for treating, preventing or ameliorating a disease, disorder or condition associated with impaired function of the enzyme 3-hydroxy-3-methyl-glutaryl-coenzyme A reductase (HMGCR), comprising administrating a therapeutically effective amount of mevalonolactone to a subject in need thereof, thereby treating, ameliorating or preventing the disease disorder or condition in the subject.

9. The method of claim 8, wherein the impaired function of the enzyme HMGCR is conversion of (3S)-hydroxy-3-methylglutaryl-CoA (HMG-COA) to mevalonic acid.

10. The method of claim 7, wherein the impaired function of HMGCR enzyme is inhibited, suppressed or arrested activity of the enzyme.

11. The method of claim 10, wherein the enzyme is inhibited or suppressed due to one or more deleterious and/or harmful, direct or indirect, exogenous effects or agents.

12. The method of claim 11, wherein the exogenous agents are one or more toxins or medications, or a combination thereof.

13. The method of claim 12, wherein the medication is at least one of a checkpoint inhibitor, immunotherapy, corticosteroids, cholesterol-lowering drug, amiodarone, colchicine, chloroquine, antivirals and protease inhibitor used in the treatment of HIV infection, omeprazole, or any combination thereof.

14. The method of claim 13, wherein the medicament is a statin.

15. The method of claim 10, wherein the enzyme is inhibited or suppressed due to one or more deleterious and/or harmful, direct or indirect, endogenous effects or events, wherein the endogenous event is one or more of: a mutation, post transcriptional modification, protein post translational modification and/or a chemical or biological agent or factor produced in the body.

16. (canceled)

17. The method of claim 15, wherein the enzyme is inhibited, hindered or suppressed due to one of more mutations in the gene that encodes the enzyme (HMGCR).

18. (canceled)

19. The method of claim 5, wherein the disease, disorder or condition is one or more of: myopathy, statin-associated muscle symptom (SAMS), immune-mediated necrotizing myopathy (IMNM), rhabdomyolysis, muscular toxicity syndrome, myalgia, sarcopenia, limb girdle muscular dystrophy (LGMD) or biallelic HGMCR mutation limb girdle muscular dystrophy (HGMCR-LGMD).

20. The method of claim 19, wherein the SAMS persists after statin cessation.

21. The method of claim 1, wherein mevalonolactone is D-mevalonolactone (R-(−)-mevalonolactone).

22. The method of claim 1, wherein mevalonolactone is administered to the subject orally.

23. A process for producing D-mevalonolactone, comprising the steps of:
(i) obtaining E. coli bacteria cells transfected with a plasmid containing the gene coding for the enzyme 3-hydroxy-3-methyl-glutaryl-coenzyme A reductase (HMGCR);
(ii) transferring the transformed bacterial cells are into a bioreactor system containing a growth medium comprising yeast extracts and one or more osmotic stress controlling agents;
(iii) growing the transformed bacteria cells until a sufficient biomass is achieved;
(iv) inducing the transformed bacteria cells to synthesize D-mevalonolactone by the addition of an inducer;
(v) separating the bacterial cells from the broth medium that contains the synthesized product; and
(vi) extracting the D-mevalonolactone via organic extraction, facilitated by (a) acidification to a pH 2 to allow conversion of mevalonate to mevalonolactone; and (b) salt-outing to remove remaining cells and proteins.

24. The method of claim 5, wherein mevalonolactone is D-mevalonolactone (R-(−)-mevalonolactone).

25. The method of claim 8, wherein mevalonolactone is D-mevalonolactone (R-(−)-mevalonolactone).

26. The method of claim 5, wherein mevalonolactone is administered to the subject orally
